(19) United States
(12) Patent Application Publication
(10) Pub. No.: US 2019/0241857 A1
(43) Pub. Date: Aug. 8, 2019

(54) DEVICE FOR CULTURING CELLS AND METHOD FOR MAKING THE SAME

(71) Applicant: Chung Yuan Christian University, Taoyuan City (TW)

(72) Inventors: Tzong-Rong Ger, Taoyuan City (TW); Tan-Yueh Chen, Taoyuan City (TW); Chao-Ming Su, Taoyuan City (TW); Yin-Chou Huang, Taoyuan City (TW); Fan Dong, Taoyuan City (TW); Yu-Chi Kuo, Taoyuan City (TW)

(21) Appl. No.: 16/261,249

(22) Filed: Jan. 29, 2019

(30) Foreign Application Priority Data
Feb. 5, 2018 (TW) 107104055

(51) Int. Cl. C12M 1/26; C12M 1/12; C12M 1/42; C12N 5/00; C12M 1/34

(52) U.S. Cl. CPC C12M 33/00; C12M 25/14; C12M 35/06; C12N 5/0068; C12N 2537/00; C12N 2523/00; C12N 2527/00; C12N 2533/30; C12N 2535/10; C12M 41/12

(57) ABSTRACT

A device and method for cultivating cells are provided, wherein a cell cultivating layer is formed on a surface of a substrate, and a temperature-responsive layer having a plurality of temperature-responsive polymer with magnetic objects is formed on a surface of the cell cultivating layer. When a controlling characteristic exerted on the temperature-responsive layer is varied, a plurality of cells can be adhered on the cell cultivating layer or detached therefrom. When the device is utilized, a temperature control step is operated to control environmental temperature at a first temperature for inducing temperature-responsive polymers becoming hydrophobic whereby the plurality of cells are adhered on the cell cultivating layer. In addition, the alternating magnetic field is utilized to rise temperature of the temperature-responsive polymers so that the plurality of cells can be kept being adhered on the cell cultivating layer at a second temperature lower than the first temperature.

This application claims the benefit of Taiwan Patent Application Serial No. 107104055, filed on Feb. 5, 2018, the subject matter of which is incorporated herein by reference.

### BACKGROUND OF INVENTION

### 1. Field of the Invention

The present invention is related to cell cultivating technology, and more particularly, to a method and device for cultivating cells through a variation of controlling characteristic that changes a status of temperature-responsive polymers having magnetic objects whereby the cell can be attached on a substrate or detached therefrom.

### 2. Description of the Prior Art

In normal organs, the homotypic cell and heterotypic cell must be grown at relative correspondence position so as to possess and demonstrate the functionality. Therefore, a subject of the cell patterning technology plays a vital role in field of biomedical engineering. The concept of cell patterning is proposed in 1960s and it is divided into active type and passive type.

In the passive type, the cells can be attached to the substrate through cell-adhesion ligand and receptor arranged on the substrate. The reaction time for attaching the cells on the substrate is long and the substrate is non-reusable. On the other hand, the active type is adapted to attach the cells on the substrate through external force such as sound wave, optical tweezers, dielectrophoresis, and so on whereby the cells can be attached to specific positions that form a pattern on the substrate. Although the active way for arranging the cells on the substrate is technically irreversible and has better reaction efficiency and short operation time, there still has limitation of application such as structure variation of cells when the cells are adhered on the porous structure formed on the substrate, and difficulty of manipulation of cell patternization. In addition, although dielectrophoresis is able to manipulate the cell group, in order to prevent the cell membrane from being damaged, a low electrically conducting environment with high frequency electricity is necessary for operating the cells. Regarding the optical tweezers, although it can be utilized to manipulate a single cell, the device is expansive and the operation power is limited in order to prevent the cell from heat damage.

In the convention techniques, such as Tang, “Temperature-Responsive Polymer Modified Surface for Cell Sheet Engineering”, Polymer, 15 Aug. 2012, 4, 1478-1498, for example, disclosed a temperature responsive material for cell cultivation on a sheet. In Tang, it disclosed that Poly(N-isopropylacrylamide) (PIPAAm), which is a well-known temperature-responsive polymer, has been grafted on tissue culture polystyrene (TCPS) surfaces through an electron beam irradiated polymerization. At 37° C., where the PIPAAm modified surface is hydrophobic, cells can adhere, spread on the surface and grow to confluence. By decreasing temperature to 20° C., since the surface turns to hydrophilic, cells can detach themselves from the surface spontaneously and form an intact cell sheet with extra cellular matrix. In addition, PIPAAm modified surfaces giving a rapid cell-sheet recovery has been further developed on the basis of the characteristic of the PIPAAm surface. The designs of temperature-responsive polymer layer have provided an enormous potential to fabricate clinically applicable regenerative medicine.

In the above-mentioned prior arts, although the device can make the cells attaching thereon, it is necessary to keep the material maintaining temperature higher than the LCST. In another words, under the normal temperature, since the temperature of the environment around the material is lower than the LCST, the cells will be detached whereby the cells can't keep the desired pattern under the normal temperature. Accordingly, there has a need for providing a device that can keep the cell being attached on the material under temperature lower than the LCST.

### SUMMARY OF THE INVENTION

The present invention provides a method and device for cultivating cells, in which a magnetic means is combined with the temperature-responsive material such that not only can the temperature-responsive material be controlled under normal LCST manipulation, but the temperature-responsive material is also manipulated to keep the cell forming a desired pattern with an interaction of alternative magnetic field under temperature lower than LCST, such as normal or room temperature, for example. Through device and method of the present invention, the problem that the cells are damaged in the conventional art can be solved and the cells can be kept patternization under temperature lower than the LCST.

The present invention provides a device and method for cultivating the cells, in addition to attach the cells onto the substrate of the device, it further to control the cell pattern on the substrate through a external magnetic field whereby the device can be utilized to simulate various kinds of organ cell so that the application field of the device can be expanded. Particularly, a film layer having structural pattern formed by magnetic material is applied to enable the cells to be attached on a desired position of the pattern and the cells attached on the desired position can be further controlled by the external magnetic field to control the attached location or area of the cell forming specific cell pattern.

The present invention provides a device and method for cultivating the cells, wherein by means of the magnetic temperature-responsive layer or magnetic temperature-responsive layer with magnetic film, the cells can be controlled to attach to or detach from the substrate under the temperature lower than the LCST without injecting magnetic objects into the cells so as to keep the cell entirety thereby expanding the application field.

In one embodiment, the present invention provides a device for cultivating cells which comprises a substrate, a cell cultivating layer, formed on a surface of the substrate, a temperature-responsive layer, formed on a surface of the cell cultivating layer, wherein the temperature-responsive layer comprises a plurality of magnetic temperature-responsive polymers, each of which comprises a temperature-responsive polymer and a plurality of magnetic objects, wherein when a controlling characteristic is varied, a plurality of cells is attached to the cell cultivating layer or detached from the cell cultivating layer.

In one embodiment, the cell cultivating layer comprises polyethylenimine, and the temperature-responsive polymer is (poly(N-Isopropylacrylamide)).

In one embodiment, a magnetic structure having a structural pattern is arranged between the cell cultivating layer and the substrate or is arranged external side of the substrate such that the temperature-responsive polymers with a plurality of magnetic objects are arranged at specific places corresponding to the structural pattern while the temperature-responsive polymers without magnetic objects are arranged on elsewhere of the cell cultivating layer. In one embodiment, the structural pattern comprises a plurality of concentric geometric patterns. In one embodiment, the temperature-responsive polymers become hydrophobic status at first temperature so that the cells can be attached to the substrate and the temperature-responsive polymers that are not corresponding to the structural pattern become hydrophilic status under a second temperature such that the cells around the hydrophilic polymers are detached while the temperature-responsive polymers that are corresponding to the structural pattern maintain hydrophobic status under the second temperature with an interaction with alternative magnetic field whereby the cells corresponding to the structural pattern can be kept being attached on the cell cultivating layer.

In one embodiment, a lateral side of the substrate has a permanent magnet with a structural pattern whereby the temperature-responsive polymers with a plurality of magnetic objects are arranged at specific places corresponding to the structural pattern while the temperature-responsive polymers without magnetic objects are arranged on elsewhere of the cell cultivating layer. In one embodiment, the structural pattern comprises a plurality of concentric geometric patterns. In one embodiment, the temperature-responsive polymers become hydrophobic status at first temperature so that the cells can be attached to the substrate and the temperature-responsive polymers that are not corresponding to the structural pattern become hydrophilic status under a second temperature such that the cells around the hydrophilic polymers are detached while the temperature-responsive polymers that are corresponding to the structural pattern maintain hydrophobic status under the second temperature with an interaction with alternative magnetic field whereby the cells corresponding to the structural pattern can be keep being attached on the cell cultivating layer.

In one embodiment, the present invention provides a method for cultivating cells, comprising steps of providing a cell cultivating device having a substrate, a cell cultivating layer formed on a surface of the substrate, a magnetic temperature-responsive layer formed on a surface of the cell cultivating layer, and a magnetic structure is arranged between the cell cultivating layer and the substrate, or externally arranged at a side of the substrate, wherein the magnetic temperature-responsive layer further comprises a plurality of magnetic temperature-responsive polymers, and each of which comprises temperature-responsive polymer and a plurality of magnetic objects, and providing an alternative magnetic field exerting on the cell cultivating device, and finally, adjusting the temperature under the LCST whereby the cells that are not corresponding to magnetic structure are detached therefrom while the cells that are corresponding to the magnetic structure are maintained to be attached on the substrate.

In one embodiment, the present invention provides a method for forming a cell cultivating device, comprising steps of forming a plurality of magnetic temperature-responsive polymers, each of which comprises a first temperature-responsive polymer having a plurality of magnetic objects, providing a substrate, forming a cell cultivating layer on the substrate, and forming a magnetic temperature-responsive layer on the cell cultivating layer by utilizing the plurality of magnetic temperature-responsive polymers thereby forming the cell cultivating device, wherein when a controlling characteristic is varied, a plurality of cells is attached to the cell cultivating layer or detached from the cell cultivating layer.

In one embodiment, before forming the cell cultivating layer on the substrate, it is further comprises a magnetic film layer on the substrate, wherein the magnetic film layer further comprises a structural pattern, wherein the plurality of magnetic temperature-responsive polymers are arranged on at least one specific position of the structural pattern after forming the magnetic temperature-responsive layer. In one embodiment, in step of forming the magnetic temperature-responsive layer, it further comprises a step of providing a mixture liquid having a plurality of temperature-responsive polymer with magnetic objects and a plurality of temperature-responsive polymer without magnetic objects, and coating the mixture on the cell cultivating layer.

All these objects achieved by device for cultivating cell and method of making the same according to the present invention are described below.

### BRIEF DESCRIPTION OF THE DRAWINGS

The present invention will now be specified with reference to its preferred embodiment illustrated in the drawings, in which:

FIG. 1 illustrates a cross-sectional view of a cell cultivating device according to one embodiment of the present invention.

FIGS. 2A and 2B respectively illustrate a variation between hydrophobic and hydrophilic of a temperature-responsive polymer under temperature higher than LCST or lower than LCST.

FIGS. 2C and 2D respectively illustrate a variation between hydrophobic and hydrophilic of a magnetic temperature-responsive polymer under temperature lower than LCST.

FIGS. 3A to 3D illustrate steps for manufacturing cell cultivating device according to one embodiment of the present invention.

FIG. 4A illustrates a cross-sectional view of a cell cultivating device according to another embodiment of the present invention.

FIG. 4B illustrates a structure of the magnetic film according to one embodiment of the present invention.

FIGS. 5A and 5B illustrate one embodiment for forming a specific cell pattern through the cell cultivating device of the present invention.

FIG. 6 illustrates an alternative embodiment of cell cultivating device of the present invention.

FIGS. 7A to 7F illustrate steps for manufacturing cell cultivating device shown in FIGS. 4A and 4B according to another embodiment of the present invention.

### DESCRIPTION OF THE PREFERRED EMBODIMENT

The invention disclosed herein is directed to device for cultivating cell and method of making the same. In the following description, numerous details corresponding to the aforesaid drawings are set forth in order to provide a thorough understanding of the present invention so that the present invention can be appreciated by one skilled in the art, wherein like numerals refer to the same or the like parts throughout.

Although the terms first, second, etc. may be used herein to describe various elements, components, modules, and/or zones, these elements, components, modules, and/or zones should not be limited by these terms. Various embodiments will now be described in conjunction with a number of schematic illustrations. The embodiments which are set forth the device for cultivating cells and method for making the same are different from the conventional approaches. Various embodiments of the application may be embodied in many different forms and should not be construed as a limitation to the embodiments set forth herein.

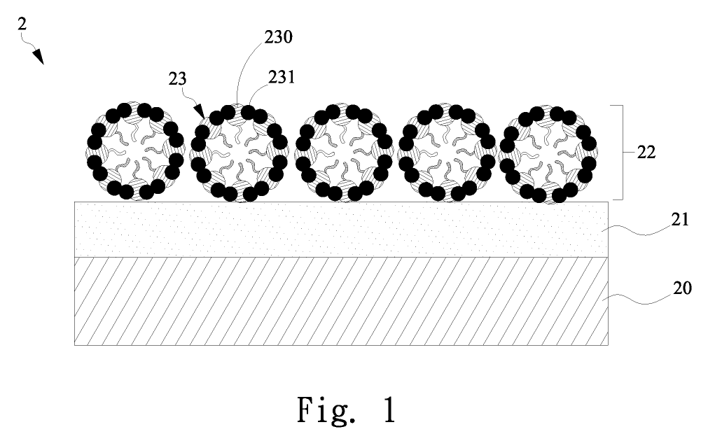

Please refer to FIG. 1 which illustrates a cross-sectional view of the cell cultivating device according to one embodiment of the present invention. In the present embodiment, the cell cultivating device 2 comprises a substrate 20, a cell cultivating layer 21 and a magnetic temperature-responsive layer 22. The material of the substrate 20 can be a hard substrate such as a glass substrate and a silicon substrate, or a flexible substrate, such as plastic substrate, for example. The substrate material is not limited to the exemplary material previously mentioned, and it is depending on the user's need. The cell cultivating layer 21 utilized to allow the cells attached thereto is formed on a top surface of the substrate 20. In one embodiment, the cell cultivating layer 21 can be, but is not limited to, polyethylenimine (PEI). It is noted that the material for forming the cell cultivating layer can be chosen according to the user's need.

On the top of the cell cultivating layer 21, the magnetic temperature-responsive layer 22 is formed thereon. The magnetic temperature-responsive layer can be converted into a hydrophobic status or hydrophilic status according to a variation of controlling characteristic such as physical characteristic or chemical characteristic. In the embodiment of the physical characteristic, it can be environmental temperature, alternative magnetic field (H_{AC}) exerting on the cell cultivating device 2, or a combination thereof. Alternatively, in the embodiment of the chemical characteristic, it can be PH value, ionic condition, isoelectric point, or material compound. The variation of PH value can control hydrophobic status or hydrophilic status of the magnetic temperature-responsive layer 22. The ionic condition, such as ionic concentration, for example, means the ion elements such as Ca²⁺ or other ions, can be utilized to control the hydrophobic status or hydrophilic status of the magnetic temperature-responsive layer 22. Likewise, the isoelectric point, or material compound can also be utilized. The magnetic temperature-responsive layer 22 comprises a plurality of magnetic temperature-responsive polymers 23, each of which further has a temperature-responsive polymer 230 and a plurality of magnetic objects 231 formed on a surface of the temperature-responsive polymer 230 or inside the temperature-responsive polymer 230. The magnetic object can be, but should not be limited to, magnetic particle, magnetic wire, magnetic tube, or magnetic cubic.

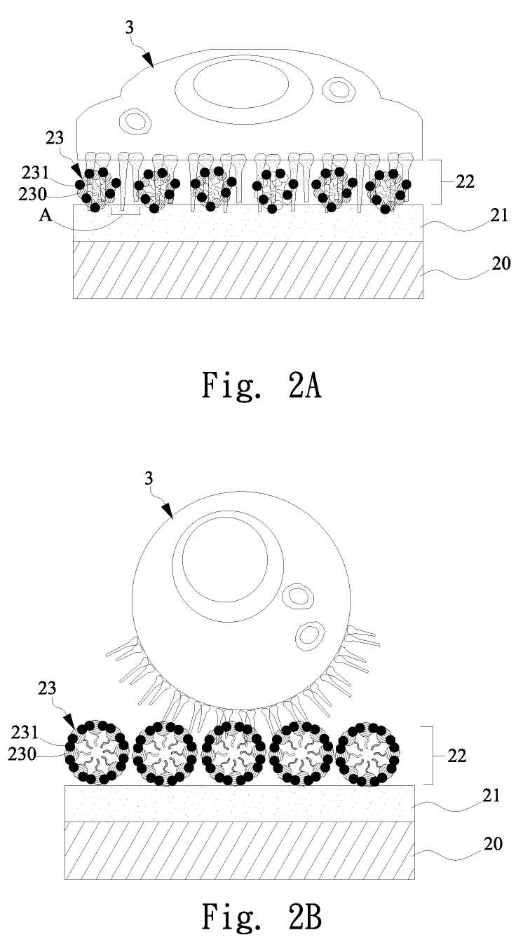

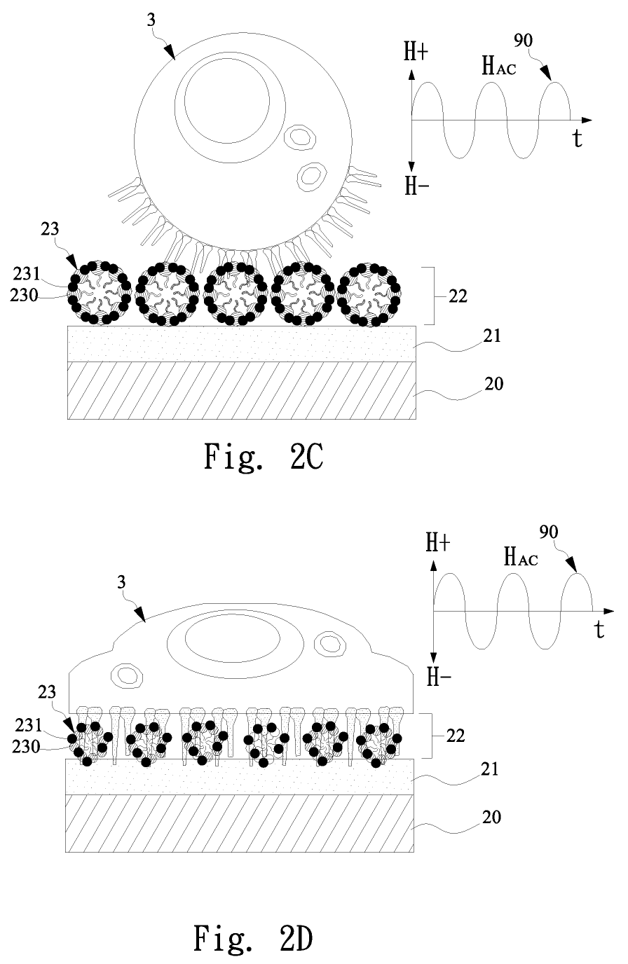

In one embodiment, the temperature-responsive polymer 230 can be, but is not limited to poly(N-Isopropylacrylamide), i.e., poly(NIPAAm). The magnetic object 231, in one embodiment, is a nano magnetic particle. Please refer to FIGS. 2A and 2B, in the present embodiment, the magnetic temperature-responsive layer 22 has a lower critical solution temperature (LCST), which is between 32˜34.4° C., for example. In the present embodiment, the LCST of the temperature-responsive polymer is 32° C. It is noted that the value or range of LCST depends on the material property of temperature-responsive polymer and it is not limited to the exemplary temperature described hereto.

In FIG. 2A, when the environmental temperature is higher than LCST, for example 37° C., the magnetic temperature-responsive polymer 23 will be contracted and converted into a hydrophobic status whereby area A of the cell cultivating layer 21 that are covered by the expanded magnetic temperature-responsive polymer 23 will be appeared. In this moment, if there has cells 3 in the environment around the cell cultivating device 2, the cells 3 will be attached to the cell cultivating layer 21. In one embodiment, the environment is a liquid environment. On the contrary, when the environmental temperature is lower than LCST, such as 28° C., for example, the magnetic temperature-responsive polymers 23 of the magnetic temperature-responsive layer 22 will be converted into hydrophilic status from the hydrophobic status, i.e. becoming expanded magnetic temperature-responsive polymer, whereby the area A is covered by the expanded polymer such that the cells are detached from the cell cultivating layer 21.

Please refer to FIGS. 2C and 2D, which respectively illustrate a variation between hydrophobic and hydrophilic of a magnetic temperature-responsive polymer under temperature lower than LCST, wherein the temperature is controlled through an alternative magnetic field 90. In FIG. 2C, when the environmental temperature is lower than LCST, such as 28° C., for example, theoretically, the magnetic temperature-responsive polymer should be converted into hydrophilic status and expands the volume thereof. However, since the alternative magnetic field 90 is exerted on the magnetic temperature-responsive polymer 23, the magnetic objects on the temperature-responsive polymer 230 will be interacted with the alternative magnetic field 90 such that temperature of the magnetic temperature-responsive polymer 23 will be increased to higher or equal to the LCST thereby enabling the magnetic temperature-responsive polymer to keep in hydrophobic status, i.e. maintaining contracted status such that the cells can be attached to the cell cultivating layer 21 or the attached cells can be maintained to keep being adhered on the cell cultivating layer 21.

Through the control of the alternative magnetic field, the cells usually are detached from the cell cultivating layer 21 under normal temperature can be kept to be attached to the cell cultivating layer 21 so that the application field with of the cell cultivating device 2 can be expanded. For example, in one embodiment of utilization, the user is not necessary to maintain the environmental temperature to keep the cells being attached to the cell cultivating layer. In another words, even if in the normal temperature that the magnetic temperature-responsive polymers should be hydrophilic, the cells still can be kept on the cell cultivating layer through an interaction between the alternative magnetic field and the magnetic temperature-responsive layer whereby the cost, including device and energy, of conventionally maintaining the environmental temperature measure to make the temperature-responsive polymer keep hydrophobic status can be saved as well as the convenience for transporting the cell cultivating device can be improved.

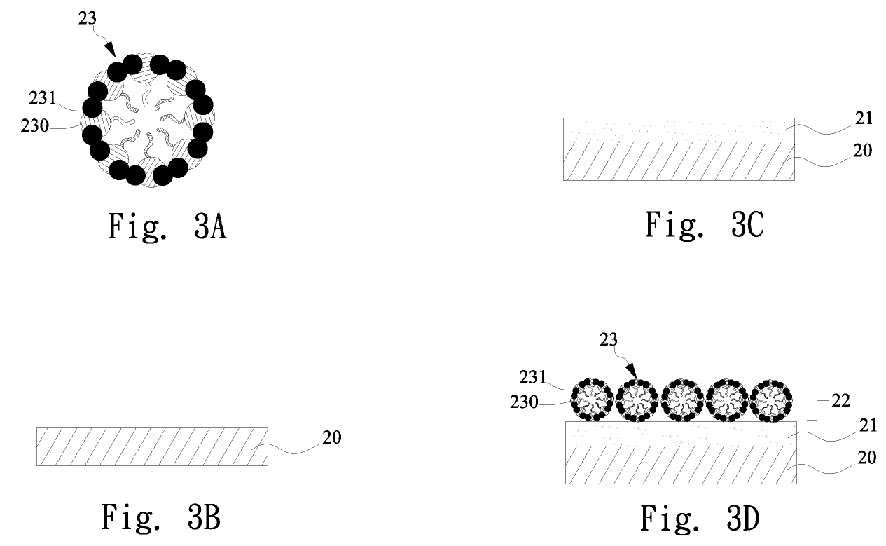

In the following, a way for manufacturing the device shown in FIG. 1 is explained. Please refer to FIGS. 3A to 3D, which illustrate steps for manufacturing cell cultivating device according to one embodiment of the present invention. At first, in step 30, an amination reaction is performed on a plurality of temperature-responsive polymer so that the plurality of temperature-responsive polymers are transformed into aminated temperature-responsive polymers. In one exemplary embodiment of this step, it further comprises dissolving the poly(NIPAAm) with N₂H₂ in the methanol (CH₃OH), heating the methanol liquid to 90° C. and refluxing the methanol liquid, cooling the methanol liquid to the room temperature, dialyzing the methanol liquid, and finally obtaining aminated poly(NIPAAm) through a freeze-drying process.

Next, a step 31 is performed to generate a temperature-responsive hydrogel material. In the step 31, it further comprises steps of dissolving 1-Ethyl-3-(3-dimethylaminopropyl)-carbodiimide (EDC) and N-hydroxysuccinimide (NHS) into deionized water respectively having 5, 10, 20 and 40 wt % CM-dextran/Fe₃O₄ wherein the EDC and NHS activate the COOH function group of CM-dextran, adding the aminated poly(NIPAAm) into the deionized water, and grafting the aminated poly(NIPAAm) with the material in the deionized water thereby forming a magnetic temperature-responsive polymers as shown in FIG. 3A. In the present embodiment, the polymer is close to the spherical shape and the magnetic nano particles are grafted on a surface of the temperature-responsive polymer.

Please refer to FIG. 3B referring to step 32, which illustrates one embodiment in accordance with the present invention. In the step 32, a substrate 20 is provided. The characteristics of the substrate 20 are as previously described and will not be described hereinafter. In the step 33, it is related to a step for forming the cell cultivating layer. Please refer to FIG. 3C, in step 33, the substrate is immersed into the solution with 1.0 wt % polyethylenimine (PEI) and is naturally dried under room temperature for forming the cell cultivating layer with 1 μm thickness in exemplary embodiment. The drying method and concentration of the PEI in the liquid are depending on the user's need and it will not be limited to the present embodiment. After that, a step 34 is performed to form a magnetic temperature-responsive layer 22 having a plurality of magnetic temperature-responsive polymer on the cell cultivating layer 21. In the step 34, a deionized water formed in FIG. 3A is dripped onto the substrate shown in FIG. 3B. The negative poly(NIPAAm) and positive PEI are tightly attracted with each other due to an electrical force generated therebetween. After a spin coating process, the poly(NIPAAm) can be uniformly coated on the substrate 20 thereby finishing poly(NIPAAm) magnetic temperature-responsive layer 22 shown in FIG. 3D.

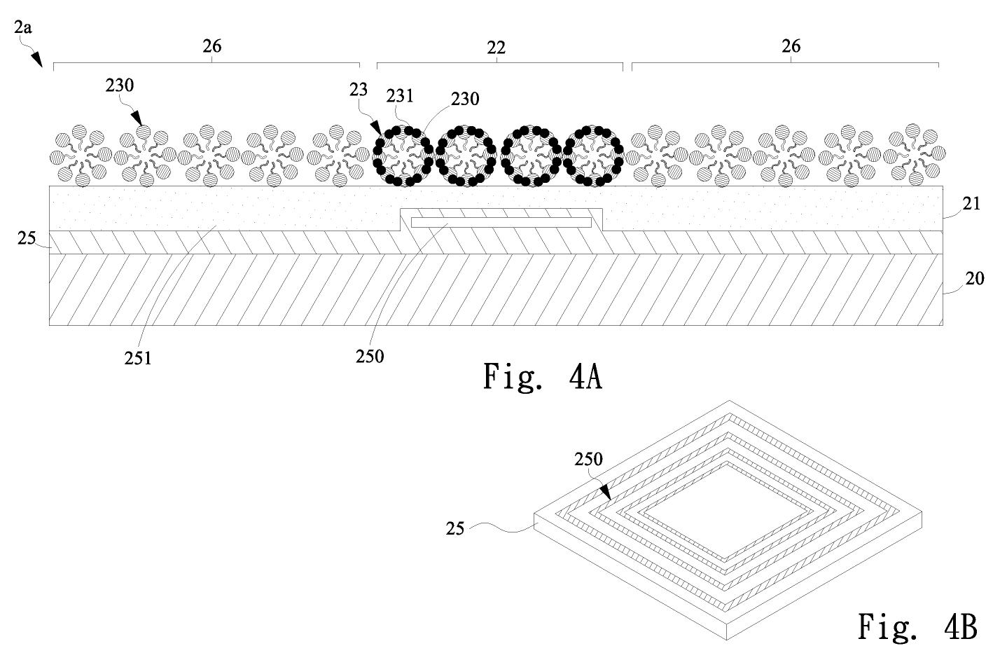

Please refer to FIG. 4A, which illustrates the cell cultivating device in accordance with another embodiment of the present invention. In the present embodiment, the cell cultivating device 2a, basically, is similar to the device shown in FIG. 1, and the difference is that a magnetic film 25 having a structural pattern 250 is formed between the cell cultivating layer 21 and substrate 20 whereby the plurality of magnetic temperature-responsive polymer 23 having magnetic objects 231 are arranged along profile of the structural pattern 250. In one embodiment, as shown in FIG. 4B, the structural pattern comprises a plurality of concentric geometric patterns. In one exemplary embodiment, the geometric pattern is a square structure. It is noted that the geometric pattern is decided according to the user's need, it is not limited to the square shape. Alternatively, in another embodiment, a plurality of temperature-responsive polymers 230 without magnetic objects can be formed on the area 251 of cell cultivating layer 21 without having the magnetic structural pattern thereby forming a temperature-responsive layer 26.

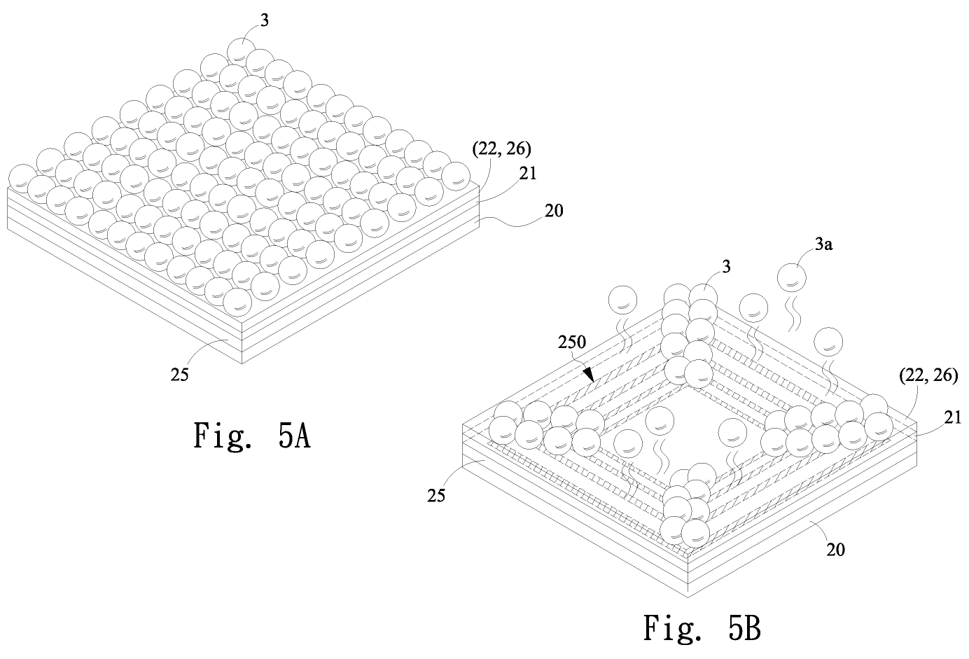

Please refer to the structure shown in FIG. 4A, which illustrates a pattern for simulating cell with specific functionality, such as liver cell and heart cell, for example, after attaching the cells on the cell cultivating device 2a. The cell simulation application can expand utilization field of the cell cultivating device for assisting the development of the medicine or treatment. Taking the structure shown in FIGS. 4A and 4B as an example, when the temperature around the cell cultivating device 2a is controlled to be higher than the LCST such as 37° C., for example, like the status shown in FIG. 2A, since the magnetic temperature-responsive polymers become hydrophobic such that the magnetic temperature-responsive polymers are contracted whereby the cells can be attached on the cell cultivating layer 21 as shown in FIG. 5A. On the contrary, when the temperature is controlled to be lower than LCST, such as 27° C., for example, the temperature-responsive polymers are expanded as the status shown in FIG. 2B, whereby the cells are detached from the cell cultivating layer 21. In the condition that the temperature is lower than LCST, when an alternative magnetic field H_{AC} is exerted on the cell cultivating device 2a, the magnetic objects of the magnetic temperature-responsive polymers will be interacted with the alternative magnetic field thereby raising the temperature such that the hydrophobic status of the magnetic temperature-responsive polymers can be maintained to keep the cells being attached on the cell cultivating layer 21.

Regarding the areas that are not corresponding to the structural pattern, i.e. without magnetic effect, since the temperature-responsive polymers are formed on those areas, the temperature-responsive polymers become hydrophilic when the temperature is lower than LCST whereby the cells originally attached on the cell cultivating layer 21 will be detached from the cell cultivating layer 21. Through the magnetic temperature-responsive polymers formed corresponding to the magnetic structural pattern, the temperature-responsive polymers formed corresponding to the areas without the magnetic structural pattern, and pulsing the LCST temperature control and alternative magnetic field interaction, the cells attached on the specific area of the cell cultivating layer 21 can be prevented from being detached, thereby forming the cell cultivating device 2a as specifically functional cells.

Next, a principle of cell arrangement is explained below. In the present embodiment, since the structural pattern comprises a plurality of concentric rectangular shape structure, the magnetic temperature-responsive polymers 23 having the plurality of magnetic objects are arranged along the profile of the structural pattern. When the temperature is lower than LCST and the alternative magnetic field H_{AC} is enabled, the cells will be attracted by the dissipative field around the corners of the structural pattern, so that the cells 3 will be concentratedly attached to the portions of the cell cultivating layer 21 where the magnetic temperature-responsive polymers are formed thereon so as to form a patterning cultivating substrate. In addition, when the cell cultivating device is arranged in the normal temperature environment without alternative magnetic field interaction, the cells can be detached completely, which is referred to another application of the present application.

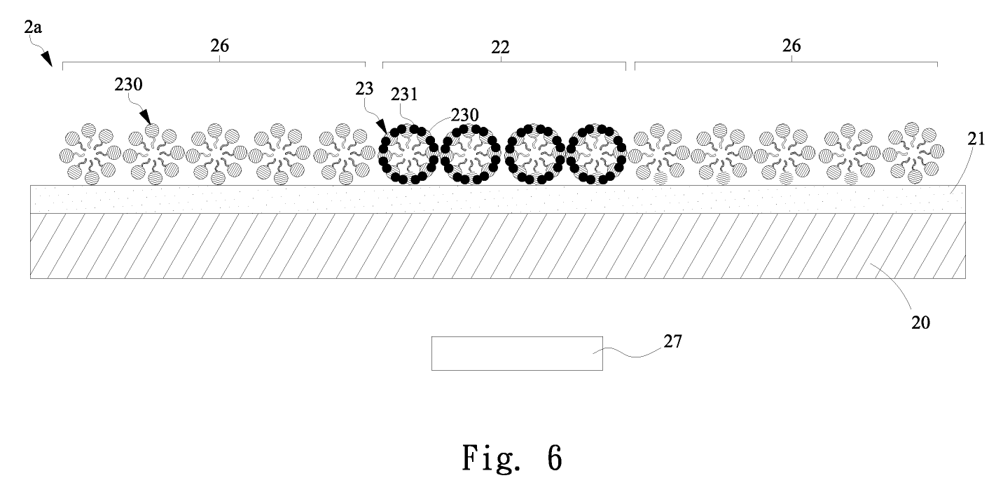

Please refer to FIG. 6, which illustrates a cell cultivating device according to another embodiment of the present application. In the present application, basically, it is the same as the structure shown in FIG. 4A. The different part is that a permanent magnet 27 having specific shape or pattern is a substitution of magnetic film 25 shown in FIG. 4A. Similarly, a plurality of magnetic temperature-responsive polymers 23 having a plurality of magnetic objects are formed on the places corresponding to the places where the permanent magnet 27 is formed, and other areas without the permanent magnet 27 have temperature-responsive polymers 230 without magnetic objects formed thereon. Regarding the cells attachment mechanism controlled by the alternative magnetic field, it is the same as previously described embodiment and will be explained hereinafter.

In the next, the method for cell cultivation of the present invention is further explained in the following. At first, step 40 is performed to provide a cell cultivating device 2a or 2b shown in FIG. 4A or FIG. 6, respectively. In the present embodiment, the cell cultivating device 2a is utilized. After that, a step 41 is performed to enable a plurality of cells to be attached on the cell cultivating device 2a, i.e. having magnetic temperature-responsive polymers formed on locations corresponding to the structural pattern of magnetic film and temperature-responsive polymers formed on the areas without the structural pattern of magnetic film, at a first working temperature. The first working temperature is higher than the LCST of the temperature-responsive polymers, and magnetic temperature-responsive polymers utilized in the cell cultivating device 2a. In the present embodiment, the first working temperature is 37° C. Under the condition of first working temperature, the cells attached on the cell cultivating device 2a is illustrated as FIG. 5A, wherein the cells 3 are completely covered on the surface having temperature-responsive polymers and magnetic temperature-responsive polymers. Thereafter, a step 42 is performed to provide an alternative magnetic field H_{AC} acting on the cell cultivating device 2a. Next, in the step 43, the first working temperature is lowered to be a second working temperature smaller than the LCST. Under the condition of step 43, the cells attached on the area having the temperature-responsive polymers are detached, while partial cells corresponding to the magnetic temperature-responsive polymers are still attached on the cell cultivating layer 21 because the temperature of the magnetic temperature-responsive polymers is still kept higher than LCST due to the interaction between the alternative magnetic field and the magnetic objects of the magnetic temperature-responsive polymers such that the cell pattern shown in FIG. 5B is formed. In one embodiment, the second working temperature is 28° C. In the FIG. 5B, when the temperature is lower than LCST and the alternative magnetic field H_{AC} is enabled, the cells will be attracted by the dissipative field around the corners of the structural pattern, so that the cells 3 will be concentratedly attached to the portions around the corners of the structural pattern where the magnetic temperature-responsive polymers are formed thereon so as to form a patterning cultivating substrate shown in FIG. 5B.

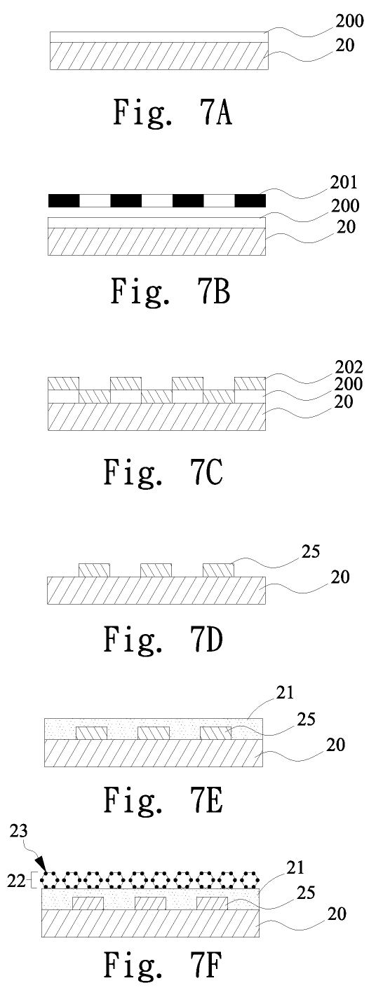

Please refer to FIGS. 7A˜7F, which illustrate an exemplary flow for forming cell cultivating device shown in FIGS. 4A and 4B. In the present embodiment, a step 50 is performed to make temperature-responsive polymers (poly(NIPAAm)). In one embodiment, NIPAAm and chain transfer agent, such as methyl 3-mercaptopropionate, for example, are added into a reaction bottle having 40 ml deionized water contained therein and nitrogen gas is then added into the bottle. After that, heat is provided to the contents inside the bottle. Next, initiator, such as potassium peroxydisulfate (KPS), for example, is added into the contents whereby a polymerization reaction is started to form the poly(NIPAAm).

After that, in the step 51, a magnetic temperature-responsive hydrogel is produced, wherein, in one embodiment, it further comprises steps of dissolving the poly(NIPAAm) with N₂H₂ in the methanol (CH₃OH), heating the methanol liquid to 90° C. and refluxing the methanol liquid, cooling the methanol liquid to the room temperature, dialyzing the methanol liquid, and finally obtaining aminated poly(NIPAAm) through a freeze-drying process. After that, further steps are proceed, which includes steps of dissolving 1-Ethyl-3-(3-dimethylaminopropyl)-carbodiimide (EDC) and N-hydroxysuccinimide (NHS) into deionized water respectively having 5, 10, 20 and 40 wt % CM-dextran/Fe₃O₄ wherein the EDC and NHS activate the COOH function group of CM-dextran, adding the aminated poly(NIPAAm) into the deionized water, and grafting the aminated poly(NIPAAm) with the material in the deionized water thereby forming a magnetic temperature-responsive polymers as shown in FIG. 3A. In the present embodiment, the polymer is close to the spherical shape and the magnetic nano-particles are grafted on a surface of the temperature-responsive polymer 230.

After that, step 52 is performed to form magnetic film with structural pattern on the substrate. The detail of the step 52 is explained in the following. At first, AZ photoresists 200 is coated on a substrate 20 illustrated as FIG. 7A. Next, as shown in FIG. 7B, reticle 201 is arranged over the photoresists 200 and a lithography process including ultraviolet light (UV) exposure, and development step is then performed to remove the photoresists at area where the ferromagnetic material will be deposited. After that, an evaporation process, such as electron beam evaporation is performed to deposit ferromagnetic material on the places having photoresist and without photoresist, thereby forming a structure shown in FIG. 7C.

After that, as shown in FIG. 7D, a lift-off process is performed to remove the photoresist with ferromagnetic material formed thereon so that the ferromagnetic film 25 having structural pattern is formed on the substrate 20, wherein the structural pattern has a plurality of concentric square structures having specific line width and spaced apart with a specific distance. In the present embodiment, the line width and the specific distance is 12 μm. After that, an external magnetic field is utilized to magnetizing the ferromagnetic film so that the structural pattern can be magnetized to dissipate magnetic field.

After forming the magnetic film, step 53 is utilized to form the cell cultivating layer on the magnetic film. In one embodiment of step 53, as shown in FIG. 7E, the substrate having magnetic film with the structural pattern is soaked into 1.0 wt % polyethylenimine (PEI) solution and are dried under room temperature naturally thereby forming a cell cultivating layer 21 having a specific thickness, such as 1 μm, for example. As shown in FIG. 7F, a step 54 is performed to coat the temperature-responsive and magnetic temperature responsive polymers on the substrate shown in FIG. 7E. In one embodiment of step 54, a spin coating process is executed after dripping a mixture solution having the hydrogel material containing the temperature-responsive polymers and magnetic temperature-responsive polymers onto the cell cultivating layer 21. During the drying process, the magnetic temperature-responsive polymers will be attracted to the positions corresponding to the structural pattern of the magnetic film, while the temperature-responsive polymers will be attracted to the places of cell cultivating layer without the structural pattern. After the drying process, a cell cultivating device as shown in FIG. 4A can be formed completely.

While the present invention has been particularly shown and described with reference to a preferred embodiment, it will be understood by those skilled in the art that various changes in form and detail may be without departing from the spirit and scope of the present invention.

What is claimed is:

1. A device for cultivating cells, comprising:
a substrate;
a cell cultivating layer, formed on a surface of the substrate; and
a temperature-responsive layer, formed on a surface of the cell cultivating layer, wherein the temperature-responsive layer comprises a plurality of magnetic temperature-responsive polymers, each of which comprises a temperature-responsive polymer and a plurality of magnetic objects;
wherein a plurality of cells are attached to the cell cultivating layer or detached from the cell cultivating layer according to a variation of at least one controlling characteristic.

2. The device of claim 1, wherein the cell cultivating layer comprises polyethylenimine, and the temperature-responsive polymer is (poly(N-Isopropylacrylamide)).

3. The device of claim 1, wherein the controlling characteristic is physical characteristic or a chemical characteristic, wherein the physical characteristic is temperature, magnetic field, or the combination of temperature and magnetic field, and the chemical characteristic is PH value, ionic condition, isoelectric point, or material compound.

4. The device of claim 1, further comprising a magnetic structure having a structural pattern, wherein the magnetic structure is arranged between the cell cultivating layer and the substrate or is arranged at external side of the substrate.

5. The device of claim 4, wherein the structural pattern comprises a plurality of concentric geometric patterns.

6. The device of claim 5, wherein the plurality of magnetic temperature-responsive polymers are arranged on at least one specific position of the structural pattern or are arranged along the structural pattern.

7. The device of claim 6, wherein the plurality of cells are attracted by a dissipative field around corners of the structural pattern, so that the cells are concentratedly attached to portions around corners where the magnetic temperature-responsive polymers are formed thereon so as to form a patterning cell cultivating substrate.

8. A method for forming a cell cultivating device, comprising steps of:
forming a plurality of magnetic temperature-responsive polymers, each of which comprises a first temperature-responsive polymer having a plurality of magnetic objects;
providing a substrate;
forming a cell cultivating layer on the substrate; and
forming a temperature-responsive layer on the cell cultivating layer by utilizing the plurality of magnetic temperature-responsive polymers thereby forming the cell cultivating device;
wherein a plurality of cells are attached to the cell cultivating layer or detached from the cell cultivating layer according to a variation of at least one controlling characteristic.

9. The method of claim 8, wherein before forming the cell cultivating layer on the substrate, it further comprises a step of forming a magnetic film layer on the substrate, wherein the magnetic film layer further comprises a structural pattern.

10. The method of claim 9, wherein the plurality of magnetic temperature-responsive polymers are arranged on at least one specific position of the structural pattern after forming the temperature-responsive layer.

11. The method of claim 9, wherein the temperature-responsive layer is formed by coating a mixed liquid comprising a plurality of second temperature-responsive polymers mixed with the magnetic temperature-responsive polymers on the cell cultivating layer wherein area corresponding to the structural pattern has the magnetic temperature-responsive polymers formed thereon while the second temperature-responsive polymers are formed on the cell cultivating layer without having the structural pattern.

12. The method of claim 8, further comprising a step of arranging a permanent magnet having a structural pattern at one side of the substrate.

13. The method of claim 11, wherein the plurality of magnetic temperature-responsive polymers are arranged on at least one specific position of the structural pattern or are arranged along the structural pattern.

14. The method of claim 12, wherein the plurality of cells are attracted by a dissipative field around corners of the structural pattern, so that the cells are concentratedly attached to portions around corners where the magnetic temperature-responsive polymers are formed thereon so as to form a patterning cell cultivating substrate.

15. The method of claim 10, wherein the controlling characteristic is physical characteristic combining temperature and magnetic field, and the magnetic temperature-responsive polymers are contracted under a first temperature whereby the cells are attached on the cell cultivating layer, while the magnetic temperature-responsive polymers corresponding to the specific position of the structural pattern maintaining contracting status under a second temperature with a variation of the magnetic field whereby the cells located at the specific position are attached by the cell cultivating layer.

16. A method for cell cultivation, comprising steps of:
providing a cell cultivating device comprising a substrate, a cell cultivating layer, formed on a surface of the substrate, a temperature-responsive layer, formed on a surface of the cell cultivating layer, wherein the temperature-responsive layer comprises a plurality of magnetic temperature-responsive polymers, each of which comprises a first temperature-responsive polymer and a plurality of magnetic objects, and a magnetic structure having a structural pattern;
enabling a plurality of cells to be attached on the cell cultivating device at a first working temperature;
providing an alternative magnetic field; and
lowering the first working temperature to a second working temperature so as to form patterning cell cultivating substrate.

17. The method of claim 18, wherein the temperature-responsive layer is formed by coating a mixed liquid comprising a plurality of second temperature-responsive polymers mixed with the magnetic temperature-responsive polymers on the cell cultivating layer wherein area corresponding to the structural pattern has the magnetic temperature-responsive polymers formed thereon while the second temperature-responsive polymers are formed on the cell cultivating layer without having the structural pattern.

18. The method of claim 16, wherein the plurality of cells are attracted by a dissipative field around corners of the structural pattern, so that the cells are concentratedly attached to portions around corners where the magnetic temperature-responsive polymers are formed thereon so as to form the patterning cell cultivating substrate.

19. The method of claim 16, wherein the magnetic structure is arranged between the cell cultivating layer and the substrate or is arranged at external side of the substrate.

20. The method of claim 16, wherein the structural pattern comprises a plurality of concentric geometric patterns.
